(19) United States
(12) Patent Application Publication
(10) Pub. No.: US 2025/0229004 A1
(43) Pub. Date: Jul. 17, 2025

(54) BIOCOMPATIBLE COMPOSITION, METHOD FOR PREPARING THE SAME, AND METHOD FOR TISSUE ADHESION

(71) Applicant: TAIPEI MEDICAL UNIVERSITY, Taipei City (TW)

(72) Inventors: Pei-Chun Wong, New Taipei City (TW); Kuan-Hao Chen, New Taipei City (TW); Wei-Ru Wang, Taipei City (TW); Chieh-Ying Chen, Keelung City (TW); Jia-Lin Wu, Taipei City (TW)

(73) Assignee: TAIPEI MEDICAL UNIVERSITY, Taipei City (TW)

(21) Appl. No.: 18/959,657

(22) Filed: Nov. 26, 2024

Related U.S. Application Data
(60) Provisional application No. 63/621,120, filed on Jan. 16, 2024.

(51) Int. Cl. A61L 27/52; A61L 27/02; A61L 27/20

(52) U.S. Cl. CPC A61L 27/52; A61L 27/025; A61L 27/20; A61L 2430/06

(57) ABSTRACT

Provided is a biocompatible composition includes a chitosan-based hydrogel and a dextran solution. Also provided is a method for preparing the biocompatible composition includes reacting the chitosan-based hydrogel with the dextran solution via a cross-linking reaction. Further provided is a method of tissue adhesion includes administering the biocompatible composition to a subject in need thereof.

### CROSS REFERENCE TO RELATED APPLICATIONS

This application claims the benefit of U.S. Provisional Application No. 63/621,120, filed on Jan. 16, 2024. The content of the application is incorporated herein by reference.

### BACKGROUND OF THE INVENTION

### 1. Field of the Invention

The present disclosure relates to a biocompatible composition, and more particularly to a biocompatible composition for tissue adhesion.

### 2. Description of the Prior Art

The meniscus is a fibrocartilaginous structure located between the distal femur and proximal tibia. The meniscus provides many biomechanical functions including load transmission, shock absorption, maintenance of joint stability, provision of joint nutrition and lubrication, and assistance to the anterior cruciate ligament (ACL) to limit tibial anterior-posterior displacement. Meniscus tears are a common clinical knee injury, which may be caused by abnormal flexion, abnormal rotation, impacts, ACL tearing, or aging, possibly leading to meniscus tearing and rupture and further causing knee osteoarthritis. The type of meniscus tear can be roughly divided into longitudinal, radial, and horizontal tears, and these types can further evolve into bucket-handle, parrot-beak, and flap tears.

Currently, meniscus-suturing surgery is the primary option for meniscus repair by arthroscopy in clinics. This surgical technique can provide better stability and high fracture strength compared to surgical staples and mini-screws. However, pressure from the sharp leading edge of sutures may cut through the intact meniscus tissue, called “suture pull-through”.

Therefore, various tissue adhesives have been developed as alternatives to traditional sutures and to overcome such problems for multiple applications. Unfortunately, these tissue adhesives still have some disadvantages, such as having cytotoxicity, unsatisfactory mechanical and adhesive properties, foreign body reactions, burning sensations in the application area, low tensile strength, and long setting times. For example, it is reported that an isocyanate-terminated amphiphilic block copolymer tissue adhesive has a satisfactory adhesive strength for torn meniscus repair, but these materials need up to 8 hr to cure completely. To the present, no suitable tissue adhesive has been developed for treating meniscal tears in the clinic.

In view of the foregoing, there is an unmet need in the art to provide a suitable tissue adhesive specific for meniscus repair treatment with satisfactory biosafety, biodegradable, sterilizable, and tissue-bonding characteristics.

### SUMMARY OF THE INVENTION

To solve the aforementioned problems, the present disclosure provides a biocompatible composition. The biocompatible composition includes a chitosan-based hydrogel and a dextran solution.

The present disclosure further provides a method for preparing the biocompatible composition of the present disclosure. The method includes reacting the chitosan-based hydrogel with the dextran solution via a cross-linking reaction.

The present disclosure also provides a method of tissue adhesion. The method includes administering the biocompatible composition of the present disclosure to a subject in need thereof.

The present disclosure will become easily understandable to those of ordinary skill in the art after reading the following detailed description of the embodiments that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating use of chitosan-based hydrogel reacted with dextran solution to form a tissue adhesive for meniscus repair and regeneration in accordance with embodiments of the present disclosure.

FIG. 2 is FTIR spectra of different groups of tissue adhesives in accordance with embodiments of the present disclosure.

Left panel of FIG. 3 is a line graph illustrating rheological properties of different groups of tissue adhesives in accordance with embodiments of the present disclosure.

Right panel of FIG. 3 is a line graph illustrating viscosity properties of different groups of tissue adhesives in accordance with embodiments of the present disclosure.

FIG. 4 is a line graph illustrating swelling ratios of different groups of tissue adhesives at 37° C. for 12 hr (n=4 per group) in accordance with embodiments of the present disclosure.

FIG. 5 is a line graph illustrating degradation behaviors of different groups of tissue adhesives immersed in simulated body fluid (Hank's solution) at 37° C. for 21 days in accordance with embodiments of the present disclosure.

Left panels of FIG. 6 are test settings of two pieces of porcine meniscus adhered with different groups of the tissue adhesives in accordance with embodiments of the present disclosure.

Right panel of FIG. 6 is a line graph illustrating shear stress of two pieces of porcine meniscus adhered with different groups the tissue adhesives in accordance with embodiments of the present disclosure.

Panels of FIG. 7 are bar graphs illustrating cell viability of SW1353 chondrocytes cultured with five different groups of tissue adhesives for 1, 3, and 7 days. (n=5 per group; ***p<0.005; N.S., not significant) in accordance with embodiments of the present disclosure.

Upper left panel of FIG. 8 is images illustrating cell migration after an initial scratch and then incubation for 24 hr in accordance with embodiments of the present disclosure.

Upper middle panel of FIG. 8 is a bar graph illustrating reduction in gap distance resulting from cell migration after 24 hr of incubation in accordance with embodiments of the present disclosure.

Lower panels of FIG. 8 are images illustrating the distance of cell migration in the microchannel on a chip platform for 48 hr by induction of different groups of tissue adhesive precipitate medium in accordance with embodiments of the present disclosure.

Upper right panel is a bar graph illustrating migration distances of SW1353 chondrocytes after 24 hr and 48 hr of incubation (n=3 per group; **p<0.01; N.S., not significant) in accordance with embodiments of the present disclosure.

Left panels of FIG. 9 are images illustrating GAG expressions from SW1353 chondrocytes after 7 days of culture with different groups of tissue adhesive precipitate medium in accordance with embodiments of the present disclosure.

Middle panel of FIG. 9 is a graph illustrating quantitation of GAG expressions from SW1353 chondrocytes after 7 days of incubation with different groups of tissue adhesive precipitate medium in accordance with embodiments of the present disclosure (n=8 per group; ****p<0.001; N.S., not significant).

Right panel of FIG. 9 is a graph illustrating quantitation of GAG expressions from SW1353 chondrocytes after 14 days of incubation with different groups of tissue adhesive precipitate medium in accordance with embodiments of the present disclosure (n=8 per group; ****p<0.001; N.S., not significant).

Upper panels of FIG. 10 are tissue profile images illustrating adhesive menisci with different groups of tissue adhesives stained by H&E staining in accordance with embodiments of the present disclosure.

Lower panels of FIG. 10 are tissue profile images illustrating adhesive menisci with different groups of tissue adhesives stained by Sirius red staining in accordance with embodiments of the present disclosure.

FIG. 11 is ToF-SIMS profile images of adhesive menisci with different groups of tissue adhesives in accordance with embodiments of the present disclosure.

Upper panels of FIG. 12 are magnetic resonance images of meniscus extrusion after adhesion with different groups of tissue adhesives in accordance with embodiments of the present disclosure (n=3 per group; **p<0.01 and ****p<0.001; N.S., not significant).

Lower left panel of FIG. 12 is a bar graph illustrating extrusion percentage of the meniscus after 2 weeks of adhesion with different groups of tissue adhesives in accordance with embodiments of the present disclosure (n=3 per group; **p<0.01 and ****p<0.001; N.S., not significant).

Lower middle panel of FIG. 12 is a bar graph illustrating extrusion percentage of the meniscus after 4 weeks of adhesion with different groups of tissue adhesives in accordance with embodiments of the present disclosure (n=3 per group; **p<0.01 and ****p<0.001; N.S., not significant).

Lower right panel of FIG. 12 is a bar graph illustrating meniscus extrusion comparison between 2 and 4 weeks after adhesion with different groups of tissue adhesives in accordance with embodiments of the present disclosure (n=3 per group; **p<0.01 and ****p<0.001; N.S., not significant).

Upper panels of FIG. 13 are repaired meniscus profile using different groups of tissue adhesives in accordance with embodiments of the present disclosure.

Lower panels of FIG. 13 are histological images of the repaired site of the meniscus in the group 3-15 and control group by H&E staining in accordance with embodiments of the present disclosure.

### DETAILED DESCRIPTION

The following embodiments are provided to illustrate the present disclosure in detail. A person having ordinary skills in the art can easily understand the advantages and effects of the present disclosure after reading the disclosure of this specification, and also can implement or apply it in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

In order that the present invention may be more readily understood, certain terms are first defined. In addition, it should be noted that whenever a value or range of values of a parameter are recited, it is intended that values and ranges intermediate to the recited values are also intended to be part of this invention.

As used herein, the singular forms “a,” “an,” and “the” include plural referents, unless expressly and unequivocally limited to one referent. The term “or” is used interchangeably with the term “and/or” unless the context clearly indicates otherwise.

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, parts, or steps, etc., and should not exclude other limitations.

As used herein, the phrase “at least one,” in reference to a list of one or more elements, should be understood to mean at least one element selected from any one or more of the elements in the list of elements, but not necessarily including at least one of each element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

As used herein, the term “about” generally referring to the numerical value meant to encompass variations of ±20%, ±10%, ±5%, ±1%, ±0.5%, or ±0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by a person having ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The numeral ranges used herein are inclusive and combinable, any numeral value that falls within the numeral scope herein could be taken as a maximum or minimum value to derive the sub-ranges therefrom. For example, the numeral range “2 min to 5 min” includes any sub-ranges between the minimum value of 2 min to the maximum value of 5 min, such as the sub-ranges from 2 min to 4 min, from 3 min to 5 min, from 3 min to 4 min and so on. In addition, a plurality of numeral values used herein can be optionally selected as maximum and minimum values to derive numerical ranges. For instance, the numerical ranges of 3 min to 4 min, 3 min to 5 min, and 4 min to 5 min can be derived from the numeral values of 3 min, 4 min, and 5 min.

The term “subject” refers to a mammal. The mammal includes, but is not limited to, humans, non-human primates, canines, felines, murines, bovines, equines, porcines, sheeps, deers, wolfs, foxes, and rabbits.

As used herein, the terms “administer,” “administering” or “administration” refer to the placement of an active ingredient into a subject systematically or topically by a method or route which results in at least partial localization of the active ingredient at a desired site to produce the desired effect. For example, the active ingredient of the present disclosure may be administered to a subject by oral administration, injection, subcutaneous administration, intramuscular administration, topical administration, or nasal administration, but the present disclosure is not limited thereto. In at least one embodiment of the present disclosure, the biocompatible composition may be injected to the subject in need during arthroscopic surgery.

As used herein, the term “tissue adhesive” is a material capable of attaching and remaining on the surface of a biological substrate with the ability to interact with biological factors. Not only do some basic properties, such as biosafety, biodegradable, sterilizability, and good tissue-bonding characteristics need to be considered when determining a proper tissue adhesive for meniscus repair, but also specific design criteria, for example, being sufficiently hydrophilic to facilitate spreading on the tissue and having good mechanical properties of a network after curing.

In at least one embodiment of the present disclosure, a chitosan hydrochloride (ChitHCl) reacted with oxidative-periodate oxidized dextran (DDA) tissue adhesive (ChitHCl-DDA) may be used, combining chitosan-based hydrogel and dextran solution (e.g., oxidative dextran) to attach to the meniscus. Viscoelastic tests, viscosity tests, lap shear stress tests, Fourier transform infrared (FTIR) spectroscopy, swelling ratio tests, and degradation behavior tests may be conducted to characterize the tissue adhesive of the present disclosure. An MTT assay, alcian blue staining, migration capacity test, cell behavior observations (e.g., chemotactic effect), and protein expression tests may be used to understand the cell viability and cell responses. Moreover, an ex vivo and an in vivo test may be used to analyze tissue regeneration and biocompatibility of the tissue adhesive of the present disclosure (e.g., ChitHCl-DDA). It is revealed that the tissue adhesive of the present disclosure (e.g., ChitHCl-DDA) provides excellent tissue adhesive strength, cell viability, and cell responses. The tissue adhesive of the present disclosure has great potential for torn meniscus tissue repair and regeneration.

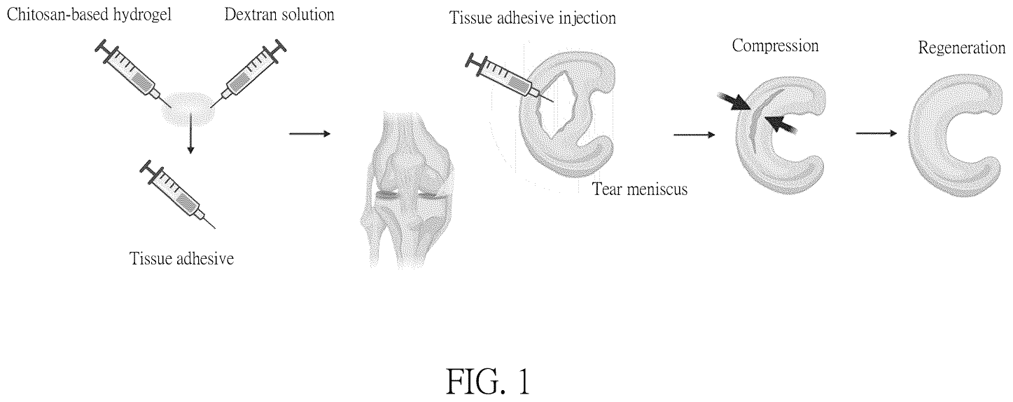

In at least one embodiment of the present disclosure, the natural polymers, chitosan and dextran, may be chosen for synthesis of a tissue adhesive of the present disclosure to repair and regenerate menisci based on the strategy of manipulating one of the biopolymers into a chemical reaction with the other resulting in the formation of a tissue adhesive, and the tissue adhesive is then administered to the subject in need thereof. For instance, as shown in FIG. 1, two syringes containing the chitosan-based hydrogel and the dextran solution, respectively, are used to mix the chitosan-based hydrogel and the dextran solution to form a cross-linked tissue adhesive outside the subject before injection. Then, the tissue adhesive is injected into the tear meniscus of the subject, and the tear meniscus is compressed. Finally, the tissue adhesive facilitates the repair and regeneration of menisci of the subject. In some embodiments, chitosan hydrochloride (ChitHCl) may be reacted with the oxidative-periodate oxidized dextran (DDA) leading to in situ gelation via Schiff's base formation. The ratio of DDA to ChitHCl may be adjusted to optimize the tissue adhesion; the mechanical properties, swelling ratio, degradation behavior, cytotoxicity, migration capacity, and protein expression of the tissue adhesive may be examined; and a porcine meniscus ex vivo test and rabbit meniscus in vivo test may be conducted.

In at least one embodiment of the present disclosure, the chitosan-based hydrogel may be crosslinked with the dextran solution.

In at least one embodiment of the present disclosure, the chitosan-based hydrogel may be about 2% to about 90% by weight of the biocompatible composition.

In at least one embodiment of the present disclosure, the chitosan-based hydrogel may be about 2% to about 90%, about 2% to about 80%, about 2% to about 70%, about 2% to about 60%, about 2% to about 50%, about 2% to about 40%, about 2% to about 30%, about 2% to about 20%, about 2% to about 10%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 90%, about 20% to about 70%, about 20% to about 60%, about 20% to about 50%, about 20% to about 40%, about 20% to about 30%, about 30% to about 90%, about 30% to about 80%, about 30% to about 70%, about 30% to about 60%, about 30% to about 50%, about 30% to about 40%, about 40% to about 90%, about 40% to about 80%, about 40% to about 70%, about 40% to about 60%, about 40% to about 50%, about 50% to about 90%, about 50% to about 80%, about 50% to about 70%, about 50% to about 60%, about 60% to about 90%, about 60% to about 80%, about 60% to about 70%, about 70% to about 90%, or about 80% to about 90% by weight of the biocompatible composition, but the present disclosure is not limited thereto. In some embodiments, the chitosan-based hydrogel may be about 2%, 5%, 10%, 15%, 20%, 25%, 30%, 35%, 40%, 45%, 50%, 55%, 60%, 65%, 70%, 75%, 80%, 85%, or 90% by weight of the biocompatible composition, but the present disclosure is not limited thereto.

In at least one embodiment of the present disclosure, the chitosan-based hydrogel may include a chitosan and a hydrochloride salt.

In at least one embodiment of the present disclosure, the chitosan may be about 2% to about 85% by weight of the biocompatible composition; and the ethanolic HCl may be about 1% to about 5% by weight of the biocompatible composition.

In at least one embodiment of the present disclosure, the chitosan may be about 2% to about 85%, about 2% to about 80%, about 2% to about 70%, about 2% to about 60%, about 2% to about 50%, about 2% to about 40%, about 2% to about 30%, about 2% to about 20%, about 2% to about 10%, about 10% to about 85%, about 10% to about 80%, about 10% to about 70%, about 10% to about 60%, about 10% to about 50%, about 10% to about 40%, about 10% to about 30%, about 10% to about 20%, about 20% to about 85%, about 20% to about 85%, about 20% to about 70%, about 20% to about 60%, about 20% to about 50%, about 20% to about 40%, about 20% to about 30%, about 30% to about 85%, about 30% to about 80%, about 30% to about 70%, about 30% to about 60%, about 30% to about 50%, about 30% to about 40%, about 40% to about 85%, about 40% to about 80%, about 40% to about 70%, about 40% to about 60%, about 40% to about 50%, about 50% to about 85%, about 50% to about 80%, about 50% to about 70%, about 50% to about 60%, about 60% to about 85%, about 60% to about 80%, about 60% to about 70%, about 70% to about 85%, or about 80% to about 85% by weight of the biocompatible composition, but the present disclosure is not limited thereto. In some embodiments, the chitosan may be about 2%, 5%, 10%, 15%, 20%, 25%, 30%, 35%, 40%, 45%, 50%, 55%, 60%, 65%, 70%, 75%, 80%, or 85% by weight of the biocompatible composition, but the present disclosure is not limited thereto.

In at least one embodiment of the present disclosure, the ethanolic HCl may be about 1% to about 5%, about 1% to about 4%, about 1% to about 3%, about 1% to about 2%, about 2% to about 5%, about 2% to about 4%, about 2% to about 3%, about 3% to about 5%, about 3% to about 4%, or about 4% to about 5% by weight of the biocompatible composition, but the present disclosure is not limited thereto. In some embodiments, the ethanolic HCl may be about 1%, 1.5%, 2%, 2.5%, 3%, 3.5%, 4%, 4.5%, or 5% by weight of the biocompatible composition, but the present disclosure is not limited thereto.

In at least one embodiment of the present disclosure, the hydrochloride salt may be about 0.1% to about 5% by weight of the biocompatible composition.

In at least one embodiment of the present disclosure, the hydrochloride salt may be about 0.1% to about 5%, about 0.1% to about 4%, about 0.1% to about 3%, about 0.1% to about 2%, about 2% to about 5%, about 2% to about 4%, about 2% to about 3%, about 3% to about 5%, about 3% to about 4%, or about 4% to about 5% by weight of the biocompatible composition, but the present disclosure is not limited thereto. In some embodiments, the hydrochloride salt may be about 0.1%, 0.5%, 1%, 1.5%, 2%, 2.5%, 3%, 3.5%, 4%, 4.5%, or 5% by weight of the biocompatible composition, but the present disclosure is not limited thereto.

In at least one embodiment of the present disclosure, the dextran solution may be about 2% to about 20% by weight of the biocompatible composition.

In at least one embodiment of the present disclosure, the dextran solution may be about 2% to about 20%, about 2% to about 10%, about 2% to about 5%, about 5% to about 20%, about 5% to about 10%, or about 10% to about 20% by weight of the biocompatible composition, but the present disclosure is not limited thereto. In some embodiments, the dextran solution may be about 2%, 5%, 10%, 15%, or 20% by weight of the biocompatible composition, but the present disclosure is not limited thereto.

In at least one embodiment of the present disclosure, the dextran solution may include a dextran, a sodium metaperiodate solution, a sodium bicarbonate solution, and a potassium iodide solution.

In at least one embodiment of the present disclosure, the dextran may be about 5% to about 10% by weight of the biocompatible composition; the sodium metaperiodate solution may be about 0.1% to about 10% by weight of the biocompatible composition; the sodium bicarbonate solution may be about 0.1% to about 5% by weight of the biocompatible composition; and the potassium iodide solution may be about 0.05% to about 20% by weight of the biocompatible composition.

In at least one embodiment of the present disclosure, the dextran may be about 5% to about 10%, about 7% to about 10%, or about 5% to about 7% by weight of the biocompatible composition, but the present disclosure is not limited thereto. In some embodiments, the dextran may be about 5%, 6%, 7%, 8%, 9%, or 10% by weight of the biocompatible composition, but the present disclosure is not limited thereto.

In at least one embodiment of the present disclosure, the sodium metaperiodate solution may be about 0.1% to about 10%, about 5% to about 10%, or about 0.1% to about 5% by weight of the biocompatible composition, but the present disclosure is not limited thereto. In some embodiments, the sodium metaperiodate solution may be about 0.1%, 0.5%, 5%, or 10% by weight of the biocompatible composition, but the present disclosure is not limited thereto.

In at least one embodiment of the present disclosure, the sodium bicarbonate solution may be about 0.1% to about 5%, about 0.5% to about 5%, about 0.1% to about 0.5%, by weight of the biocompatible composition, but the present disclosure is not limited thereto. In some embodiments, the sodium bicarbonate solution may be about 0.1%, 0.5%, 0.75%, 1%, 1.5%, 2%, 2.5%, 3%, 3.5%, 4%, 4.5%, or 5% by weight of the biocompatible composition, but the present disclosure is not limited thereto.

In at least one embodiment of the present disclosure, the potassium iodide solution may be about 0.05% to about 20%, about 0.05% to about 15%, about 0.05% to about 10%, about 0.05% to about 5%, about 5% to about 20%, about 5% to about 15%, about 5% to about 10%, about 10% to about 20%, about 10% to about 15%, or about 15% to about 20% by weight of the biocompatible composition, but the present disclosure is not limited thereto. In some embodiments, the potassium iodide solution may be about 0.05%, 0.1%, 0.5%, 1%, 5%, 10%, 15%, or 20% by weight of the biocompatible composition, but the present disclosure is not limited thereto.

In at least one embodiment of the present disclosure, the ratio of dextran solution to sodium metaperiodate solution may be about 1:0.1 to about 1:8.

In at least one embodiment of the present disclosure, the cross-linking reaction may have a gelation time of about 0.5 min to about 10 min.

In at least one embodiment of the present disclosure, the method of the present disclosure may further include preparing the chitosan-based hydrogel by mixing a chitosan powder and an ethanolic HCl.

In at least one embodiment of the present disclosure, the method of the present disclosure may further include preparing the dextran solution by mixing a dextran, a sodium metaperiodate solution, a sodium bicarbonate solution, and a potassium iodide solution.

In at least one embodiment of the present disclosure, the biocompatible composition may be topically administered to the subject.

In at least one embodiment of the present disclosure, the biocompatible composition may be topically administered to the cartilage tissue of the subject.

In at least one embodiment of the present disclosure, the cartilage tissue may be a fibrocartilaginous structure.

### EXAMPLES

Exemplary embodiments of the present disclosure are further described in the following examples, which should not be construed to limit the scope of the present disclosure.

### Materials and Experimental Procedures

### Study Design

The objective of the present disclosure is to create a biocompatible tissue adhesive for meniscus tear repair and regeneration as an alternative to meniscus sutures. The tissue adhesive of the present disclosure combines a chitosan-based hydrogel and dextran solution in a specific mixed ratio. First, five candidates of tissue adhesive are selected according to the adhesive property (e.g., adhesive strength) thereof. Second, material properties, biocompatibility, cell responses, ex vivo tests and in vivo tests of the tissue adhesive then are assessed.

### Synthesis of the Tissue Adhesive

The tissue adhesive of the present disclosure (e.g., ChitHCl-DDA) is a combination of a chitosan-based hydrogel and dextran solution in different proportions.

### Preparation of Chitosan-Based Hydrogel

The chitosan-based hydrogel of the present disclosure is prepared according to the method described by Austin and Sennett (Austin, P. R. S., S. in Chitin in Nature and Technology (ed R.; Jeuniaux Muzzarelli, C.; Gooday, G. W.) 279-286 (Plenum Press, 1986)). Briefly, 10 g of chitosan powder is first dispersed in 100 ml of 60% ethanolic HCl and stirred at room temperature. After 3 hr, a hydrochloride salt is generated, and is washed and filtered by an acetone-water mixture and dialyzed in deionized (DI) water for several days. After freeze-drying, the final product was harvested and stored at 4° C. until use. The concentration of the chitosan-based hydrogel is set to 2 wt % to 45 wt % for further use.

### Preparation of Dextran Solution

Briefly, 5 g of dextran was dissolved into 100 ml of DI water, and different ratios of a sodium metaperiodate solution (50 mg/ml) (1:4, 1:2, 1:1, and 1:0.5 (dextran solution:sodium metaperiodate solution), respectively labeled DDA 1, DDA 2, DDA 3, and DDA 4) are added and stirred at room temperature in the dark for 4 hr. Subsequently, 200 ml of a 10 wt % sodium bicarbonate solution is added for neutralization, 40 ml of potassium iodide solution is added to liberate the iodine, and the solution is kept in the dark for 15 min. The solution is then dialyzed in DI water for 72 h until the DI water is periodate-free. Finally, the dialysate is freeze-dried to obtain oxidized product. Different oxidation extents of dextran solutions are set as 20 groups which are prepared with DI water and are described in Table 1.

TABLE 1
Experimental design

Type of DDA

| Concentration | DDA 1 | DDA 2 | DDA 3 | DDA 4 |
|---|---|---|---|---|
| 1 (10 mg/ml) | 1-1 | 2-1 | 3-1 | 4-1 |
| 2 (20 mg/ml) | 1-2 | 2-2 | 3-2 | 4-2 |
| 5 (50 mg/ml) | 1-5 | 2-5 | 3-5 | 4-5 |
| 10 (100 mg/ml) | 1-10 | 2-10 | 3-10 | 4-10 |
| 15 (150 mg/ml) | 1-15 | 2-15 | 3-15 | 4-15 |

Concentration: Final concentration of the dextran solution

### Preparation of Tissue Adhesive

In at least one embodiment of the present disclosure, 2 wt % to 45 wt % chitosan-based hydrogel is prepared with DI water to react with DDA with different oxidation percentages according to the grouping in Table 1 to form cross-linked tissue adhesives.

### Adhesive Properties of Tissue Adhesive

The adhesive properties of ChitHCl-DDA tissue adhesives are determined by a modified lap shear stress test according to the ASTM F2255-05 standard. Briefly, porcine meniscus tissue is cut around the radius of the meniscus at a thickness of 2 mm and placed in phosphate-buffered saline (PBS). The tissue adhesive of the present disclosure (e.g., ChitHCl-DDA) (10 μl) is placed on two sides of the two meniscus tissues surfaces that overlapped with a 5×5-mm bonding area. Specimens are placed at room temperature for 15 min. The test is carried out with a universal test system (Hung Ta, HT2402, Taipei, Taiwan) at a tensile speed of 1 mm/min.

### Characterization of Tissue Adhesive

Fourier transform infrared (FTIR) spectroscopy (Nicolet Summit Pro, ThermoFisher Scientific, Pittsburgh, PA, USA) is used to observe and characterize compositions of the tissue adhesive of the present disclosure (e.g., ChitHCl-DDA).

### Viscoelastic and Viscosity Tests of Tissue Adhesive

Dynamic viscoelastic properties of the tissue adhesive of the present disclosure (e.g., ChitHCl-DDA) is analyzed with a rheometer (MCR 302, Anton-Paar, Graz, Austria) and a 10-mm-diameter measurement plate, and the measurement temperature is set to 37° C. by a temperature controller. Storage moduli (G′) and loss moduli (G″) of tissue adhesives of groups 2-5, 2-15, 3-5, 3-10, and 3-15 are measured in frequency sweep mode. The angular frequency is set to 0.1˜100 rad/s, and the shear strain is set to 0.5%.

### Swelling Ratios Tests of Tissue Adhesive

Tissue adhesives are made into cylinders with dimensions of 8 mm in diameter and 3 mm thick. The initial sample weight is measured and recorded before immersion. Then, a sample is immersed into simulated body fluid (Hank's solution) at 37° C. At 0, 2, 4, 6, 8, 12, and 24 hr, the sample weight and the swelling ratio of the tissue adhesive are measured and calculated, respectively.

### Degradation Behavior of Tissue Adhesive

Weight changes of the tissue adhesives of the present disclosure (e.g., ChitHCl-DDA) are used to evaluate the degradation behavior. First, the tissue adhesive is freeze-dried, and the initial weight is measured. After that, the sample is immersed in simulated body fluid (Hank's solution) at 37° C. At 3, 7, 14, and 21 days, the sample is freeze-dried and the weight is measured again at various time points. The remaining weight is calculated according to the following equation:

$$Remaining weight ( \% ) = \frac{{W}_{d}}{{W}_{i}} \times 100 \% , \qquad ( equation 1 )$$

where W_{d} is dry sample weight at the respective period and Wᵢ is initial sample dry weight.

### Preparation of Precipitate Medium

Different groups of the tissue adhesive precipitate media are prepared for the MTT assay (cell viability test), migration capacity test, alcian blue staining (glycosaminoglycan (GAG) synthesis test), and protein expression tests. The tissue adhesives are immersed in Leibovitz's L-15 medium for 7 days at a concentration of 10 mg/ml. After 7 days, the precipitate medium is collected into another tube and stored at 4° C. until use.

### Cell Viability of SW1353 Chondrocytes

SW1353 chondrocytes are used to analyze the biocompatibility (i.e., cell viability) of different groups of tissue adhesives by an MTT assay. SW1353 chondrocyte suspensions (100 μl) are added to a 96-well culture plate at a cell density of 3000 cells/well for 24 h at 37° C. in a 5% CO₂ atmosphere. Then, standard culture medium is replaced by undiluted precipitate medium and incubated in the same environment for 1, 3, and 7 days. Subsequently, 10 μl of the MTT solution (Invitrogen, Carlsbad, CA, USA) is gently added to each well and incubated for 3 h. After that, 100 μl of dimethyl sulfoxide (DMSO) is added, and the optical density is detected using an enzyme-linked immunosorbent assay (ELISA) reader at a wavelength of 560 nm (Multiskan FC; Thermo, Waltham, MA, USA).

### Migration Capacity Test of SW1353 Chondrocytes Through a Scratch Assay

The migratory capacity of SW1353 chondrocytes is evaluated with a scratch assay. A suspension (5000 cells/well) of SW1353 chondrocytes is seeded into a 24-well culture plate and incubated for 24 h. After that, a straight line is scratched along the monolayer with a 1000-μl pipette tip. Cell debris is removed with PBS and finally replaced by ChitHCl-DDA precipitate medium for further incubation for 24 hr. Cells are observed, and images captured with an optical microscope (CKX53; Olympus, Tokyo, Japan). The distance that cells traveled is measured with ImageJ software (version 1.53K, National Institutes of Health, Bethesda, MD, USA).

### Chemotactic Effect of Tissue Adhesive on SW1353 Chondrocytes Through a Channel Test

The chemotactic effect of SW1353 chondrocytes is analyzed by a channel test. 3D printed molds are designed with Shapr3D modeling software and printed with a stereolithographic (SLA) 3D printer (da Sonic Mini; Phrozen Technology, Hsinchu, Taiwan). Polydimethylsiloxane (PDMS, 1:10 crosslinker:base ratio) replica devices are cast from 3D printed molds. After plasma cleaning, PDMS replicas are bonded to glass coverslips to form a microchannel platform. After the cleaning and sterilization process, 10 μl of a SW1353 chondrocyte suspension is seeded into the port at one side of the channel and incubated for 24 hr. After cell attachment, cells are stained with Hoechst 33342 (Sigma-Aldrich; St. Louis, MO, USA), a DNA-binding fluorescent dye. Additionally, 10 μl of the tissue adhesive of the present disclosure (e.g., ChitHCl-DDA) is added to the other side of the channel. After 30 min of gelation, 10 μl of culture medium is placed in the channel and incubated again. Migrating cells in the channel are captured with a fluorescence microscope (Revolve; ECHO, San Diego, CA, USA) in a consistent position at 0, 24, and 48 hr after cell seeding, and the migrating distance is measured with ImageJ software.

### GAG Synthesis Test of SW1353 Chondrocytes

GAG synthesis test of SW1353 chondrocytes stimulated with different groups of the tissue adhesive of the present disclosure (e.g., ChitHCl-DDA) is carried out by alcian blue staining. For alcian blue staining, briefly, SW1353 chondrocyte suspensions are seeded into a 48-well culture plate and incubated for 24 hr in an incubator at 37° C. in a 5% CO₂ atmosphere. After cell attachment, the medium is replaced with different groups of precipitate medium for incubation. After 7 and 14 days, cells are fixed with 4% paraformaldehyde and stained with an alcian blue solution. After 3 hr of incubation, the staining solution is removed, and the culture plate is rinsed with DI water. Images are first captured with an optical microscope (CKX53; Olympus, Japan). Then, 100 μl of DMSO is added to dissolve the formazan crystals, and the formazan crystals are quantitated on an ELISA reader at a wavelength of 600 nm (Multiskan FC; Thermo, US).

### Torn Meniscus Repair and Regeneration of a Porcine Model by an Ex Vivo Test

Porcine menisci are obtained from the market and aseptically harvested. Menisci are rinsed three times with PBS supplemented with a 2% antibiotic/antimycotic solution. The menisci are then cut into several pieces and divided into five groups, including groups 2-5, 2-15, 3-5, 3-10, and 3-15 groups. The thickness of the meniscus pieces is 5 mm. A defect is created according to the poor clinical outcomes. A 6-mm longitudinal (vertical) tear is created with a scalpel. Then, a different type of tissue adhesive is added, and the tear is sutured with 2-0 nylon sutures according to the grouping. After that, samples are placed in six-well culture plates, and 3 ml of DMEM/F-12 medium is added for culturing and to prevent the samples from drying out. All samples are placed in an incubator at 37° C. with a 5% CO₂ atmosphere, and the medium is replaced every 3 days. After 30 days of incubation, a histological analysis is used to evaluate the situation of meniscus tissue healing with hematoxylin and eosin (H&E) and Sirius red stains. In addition, time-of-flight secondary ion mass spectrometry (ToF-SIMS; PHI TRIFT IV, ULVAC-PHI, Kanagawa, Japan) is used to analyze the profile between the meniscus tissue-tissue adhesive-meniscus tissue. ToF-SIMS-positive ion mass spectra are detected as distinct signals which are displayed and corresponded to different fragments of collagen components, such as glycine, proline, and hydroxyproline.

### In Vivo Test

### Experimental Design

All experimental protocols are followed in compliance with the Taiwanese Animal Protection Act. Animal procedures are reviewed and approved by the Institutional Animal Care and Use Committee (IACUC) of Taipei Medical University (approval no. LAC-2021-0335; 12.30.2021). Eighteen male New Zealand white rabbits with a mean body weight (BW) of 3±0.5 kg are randomly divided into six groups: groups 2-5, 2-15, 3-5, 3-10, 3-15, and control group (blank). All animals are sacrificed after 4 weeks of the experiment. There are three animals in each subgroup.

### Surgical Method

Animal surgery is performed under general anesthesia induced by an intramuscular injection of tiletamine and zolazepam (15 mg/kg BW; Virbac, Carros, France), as well as xylazine hydrochloride (12 mg/kg BW; Bayer, Leverkusen, Germany). In a single knee joint, a lateral meniscectomy is performed, and a total defect is created with a surgical blade. Then, different tissue adhesives are injected into the defect sites according to the experimental design. After the operation, rabbits are placed in independent cages and allowed unfettered movement. An antibiotic (enrofloxacin; 12 mg/kg BW; Bayer) and analgesic (ketoprofen; 12 mg/kg BW; Nang-Kuang Pharmaceutical, Taipei, Taiwan) are given before surgery and continued for 5 days to reduce infection and pain.

### Magnetic Resonance Imaging (MRI) Scans and Analysis

Two weeks and four weeks after the operation, all animals are scanned, and images are captured with a magnetic response scanning instrument (7T PharmaScan, Bruker, Billerica, MA, USA). The meniscal extrusion of different groups of tissue adhesive is observed and measured by ImageJ software according to the following equation at different time periods, grouped, and compared with the untreated group (i.e., control group):

$$Relative percentage of extrusion ( \% ) = \frac{Meniscal body extrusion}{Meniscus coronal width} \times 100 \% \qquad ( equation 2 )$$

### Histological Observations

After magnetic resonance scanning, meniscus samples are carefully isolated, fixed with 10% formalin for 5 days, and decalcified with a decalcification solution (10% formic acid and 10% HCl with distilled water) until totally decalcified. Meniscus samples are rinsed with distilled water to remove the decalcification solution and immersed in gradient alcohol (70%, 80%, 95%, and 99% alcohol for 2 h each) for the dehydration process. Meniscus samples are cleaned with xylene and immersed in paraffin for 2 hr before being embedded in a paraffin block. Finally, meniscus samples are sectioned and stained with H&E, and the meniscus adhesion site is visualized.

### Statistical Analysis

All results in the present study are presented as the mean±standard deviation (SD). All statistical analyses are performed with SPSS 20 (vers. 20.0, IBM, Armonk, NY, USA). Data are analyzed with a one-way analysis of variance (ANOVA), followed by a post-hoc Tukey's test and independent-sample t-test. Statistical significance is set at p<0.05.

### Results

### Gelation of ChitHCl Via DDA Cross-Linking

As an oxidant, dextran solution (i.e., DDA) can be cross-linked to chitosan-based hydrogel (i.e., ChitHCl) to form a typical hydrogel with adhesive properties. The gelation time of the tissue adhesive of the present disclosure (e.g., ChitHCl-DDA) is measured when the chitosan-based hydrogel (i.e., ChitHCl) and dextran solution (i.e., DDA) mixture stopped flowing in the 1.5-ml Eppendorf tube. Table 2 demonstrates gelation times of all tissue adhesive (e.g., ChitHCl-DDA) groups of the present disclosure. The tissue adhesive (e.g., ChitHCl-DDA) with a gelation time of 0.5 min to 10 min is selected as a candidate for further examination to prevent the formation of inappropriate hydrogels due to either a too-slow or too-fast gelation rate. For different concentrations of the DDA 1 group, the tissue adhesives have no successful cross-linking. On the other hand, gelation times with different concentrations of the DDA 4 group are too fast. Groups 2-5, 2-15, 3-5, 3-10, and 3-15 presented suitable gelation times within 0.5 min to 10 min and are selected as candidates.

TABLE 2
Gelation times (second(s)) of chitosan hydrochloride (ChitHCl) reacted
with oxidative-periodate oxidized dextran under different groups
of DDA at different concentrations (ratio of 10:1, ChitHCl:DDA)

Type of DDA

| Concentration | DDA 1 | DDA 2 | DDA 3 | DDA 4 |
|---|---|---|---|---|
| 1 (10 mg/ml) | Failed | Failed | Failed | Failed |
| 2 (20 mg/ml) | Failed | Failed | 33 ± 1 | 33 ± 2 |
| 5 (50 mg/ml) | Failed | 280 ± 4 | 201 ± 2 | 35 ± 1 |
| 10 (100 mg/ml) | Failed | 76 ± 1 | 155 ± 8 | 95 ± 6 |
| 15 (150 mg/ml) | Failed | 185 ± 6 | 262 ± 6 | 107 ± 1 |

Concentration: Final concentration of the dextran solution

### Characterization of Tissue Adhesive

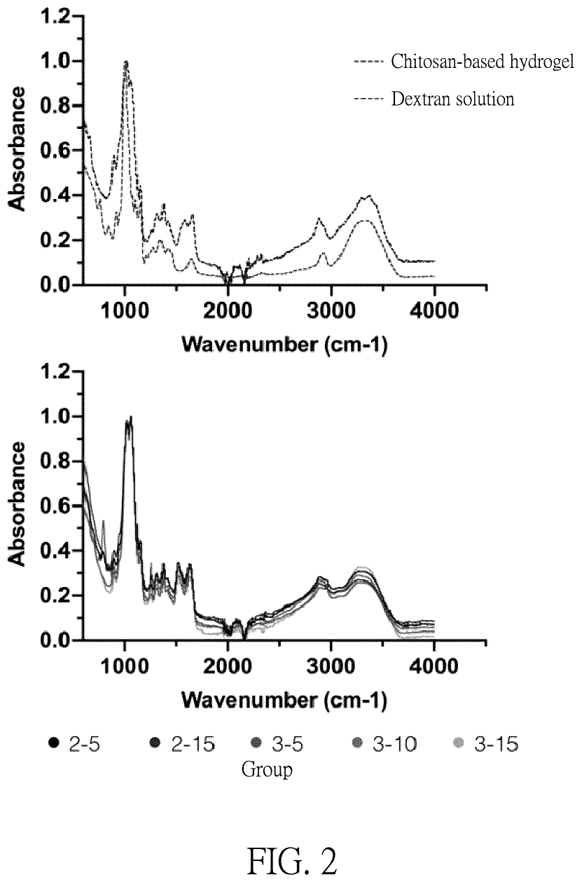

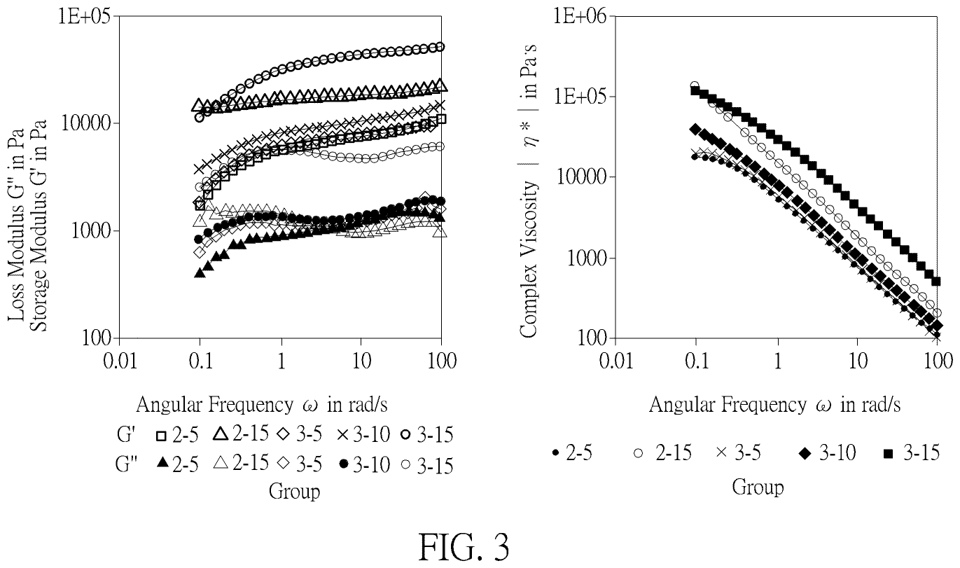

FIG. 2 shows that chitosan-specific FTIR peaks at 1598 and 3447 cm⁻¹ and dextran-specific FTIR peaks at 1019 and 2935 cm⁻¹. All the tissue adhesives of the present disclosure (e.g., ChitHCl-DDA) present the peaks in FTIR profiles thereof. FTIR spectroscopy confirmed the success of conjugation between ChitHCl and DDA.

### Viscoelastic and Viscosity Tests of Tissue Adhesive

The viscoelastic and viscosity tests of different groups of the tissue adhesives of the present disclosure are measured by a strain sweep rheological analysis after gelation. In the left panel of FIG. 3, the storage modulus (G′) in all groups is higher than the loss modulus (G″), indicating that a stable and permanent gel network with an elastic nature (solid-like) dominates over the gel viscous nature (solid-like). Comparing the G′ and G″ values of groups 2-5, 2-15, 3-5, 3-10, and 3-15, groups with higher DDA concentration exhibit higher G′ and G″ values, which means more cross-linking networks. Moreover, higher DDA concentrations of the tissue adhesive of the present disclosure also produce higher viscosity (right panel of FIG. 3). The complex viscosity of all groups of tissue adhesive exhibits large decreases with an increasing angular frequency, demonstrating that the tissue adhesives of the present disclosure are highly shear thinned.

### Swelling Ratios of Tissue Adhesive

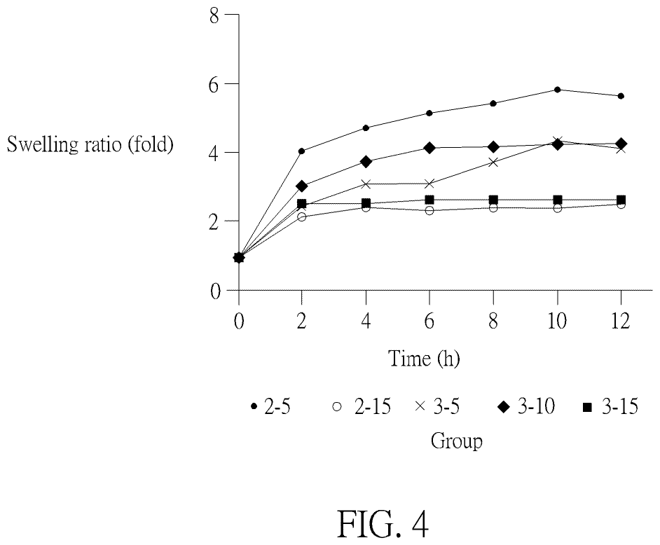

Swelling ratios of groups 2-5, 2-15, 3-5, 3-10, and 3-15 are tested for 12 hr at 37° C. (FIG. 4). In the first 2 hr, all groups continually swelled; after that, swelling ratios of groups 2-15 and 3-15 gradually stabilized. Moreover, the other groups become stable starting from 6 hr. Groups with higher concentrations of DDA (groups 2-15 and 3-15) exhibit around 2-fold lower swelling degrees compared with the others. However, group 2-5 (with higher oxidation and a lower-concentration DDA solution) shows the highest swelling ratio of around 6-fold compared with the others. The relationship indicates that the swelling ratio of the tissue adhesives of the present disclosure is related to the polymer content in the system. For meniscus tissue adhesive application, the swelling ratio must be as small as possible to maintain a stable shape to focus on localization at the tear site. Swelling of the tissue adhesive decreases the strength of the seal and can further lead to surgery failure.

### Degradation Behavior Tests of Tissue Adhesive

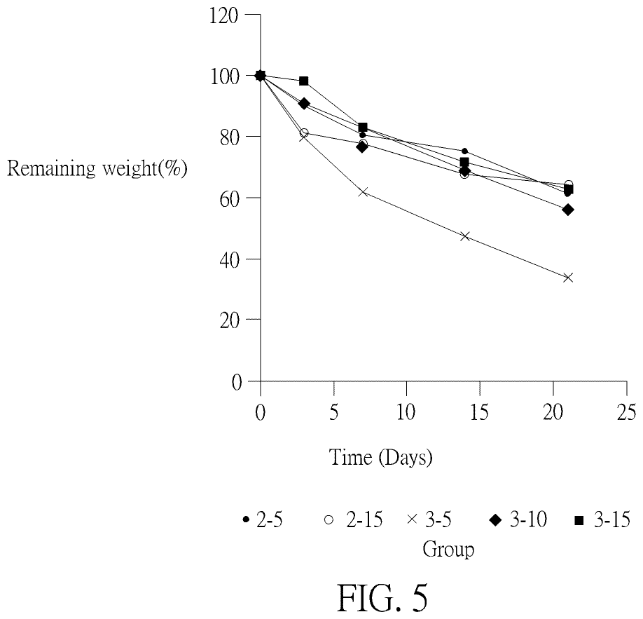

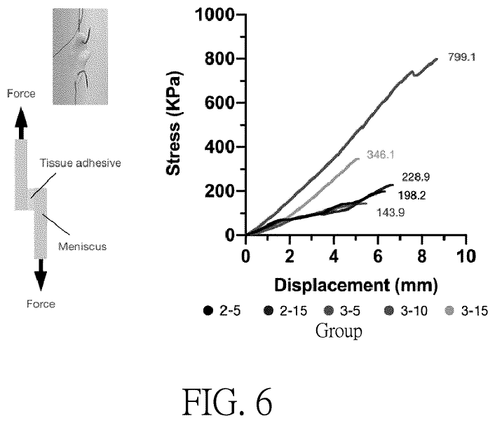

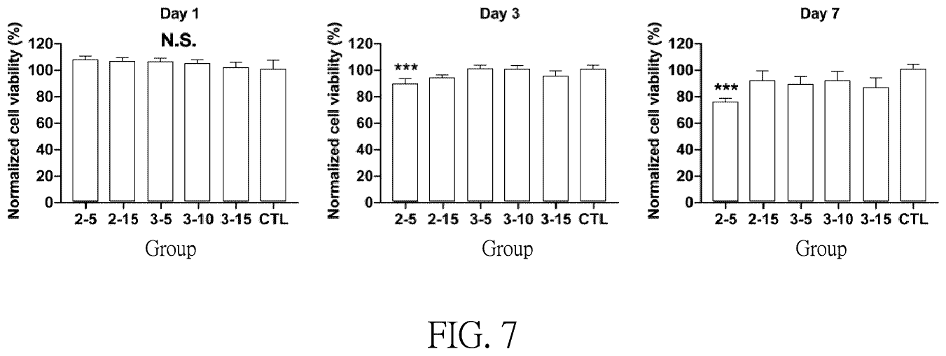

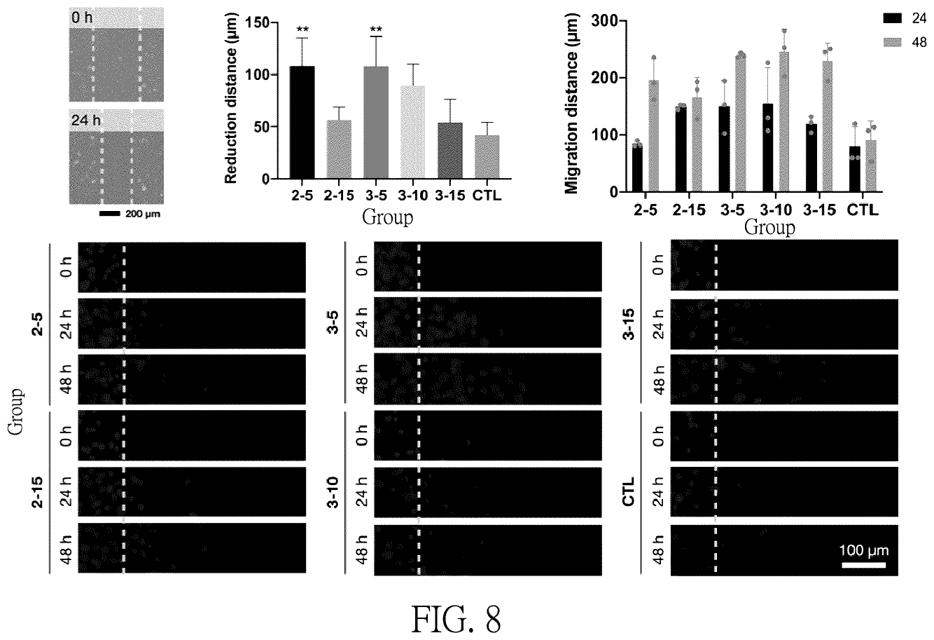

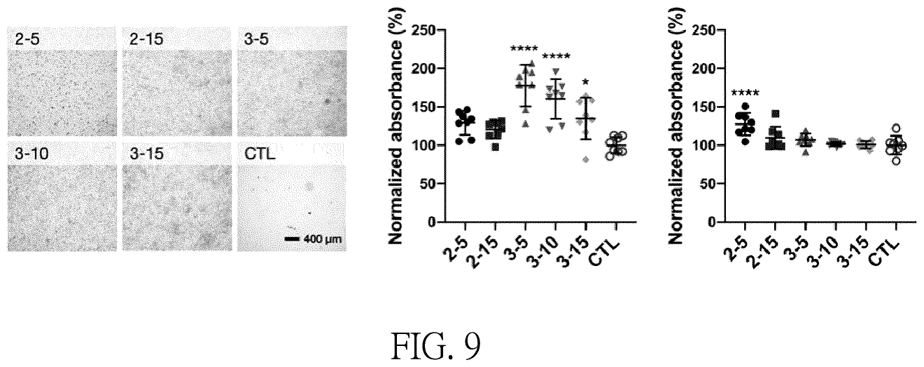

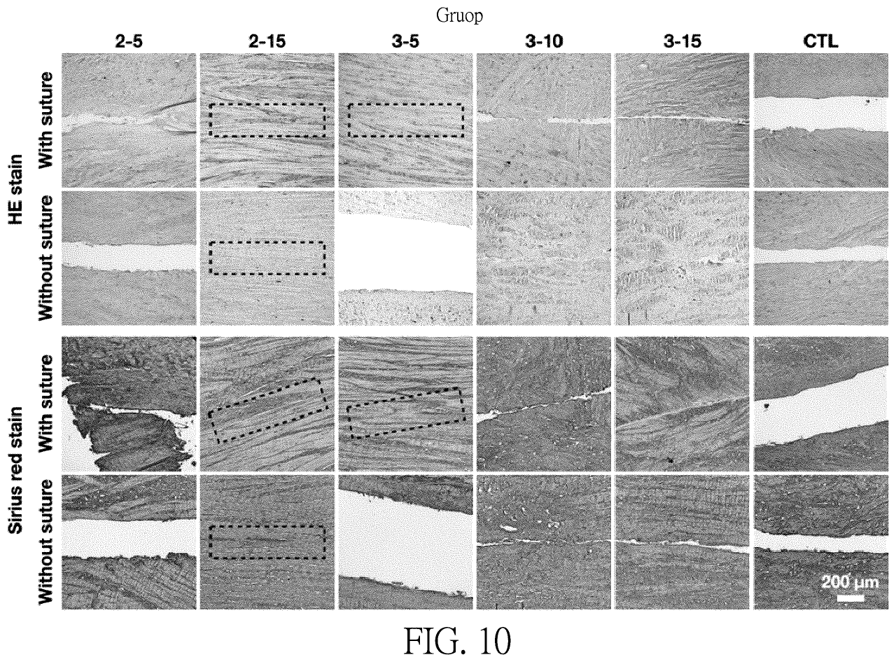

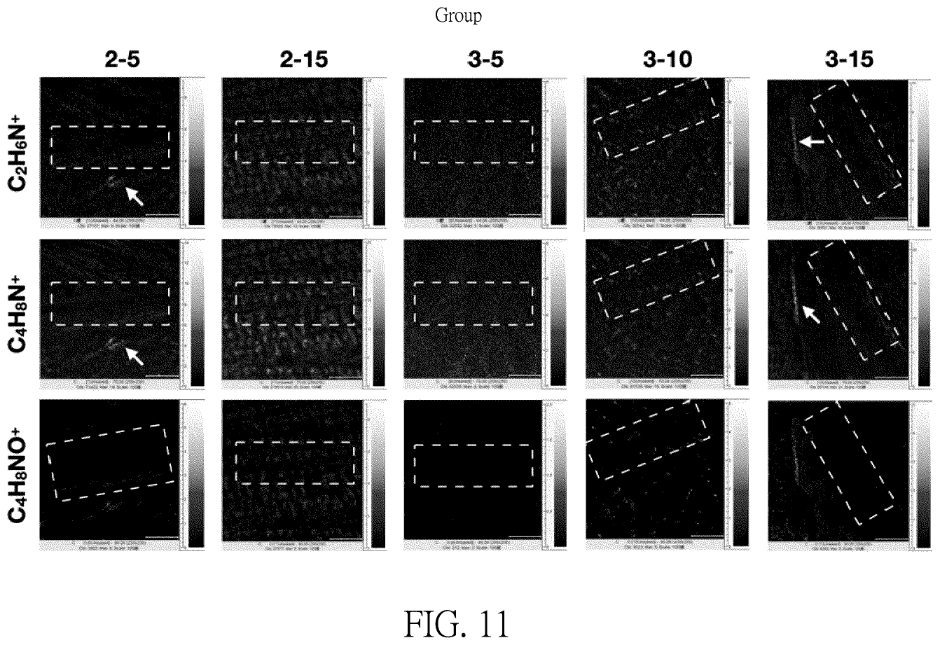

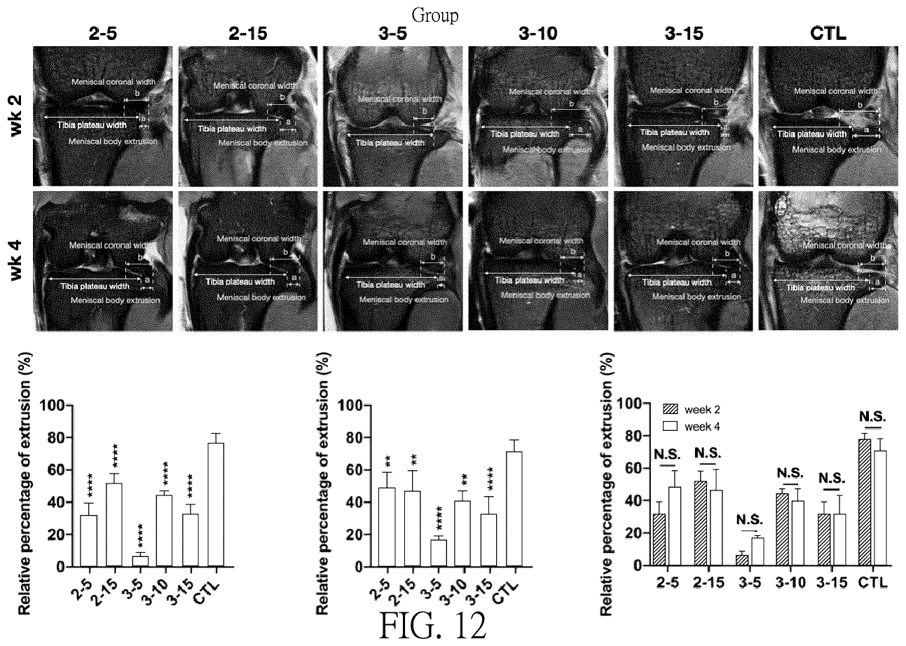

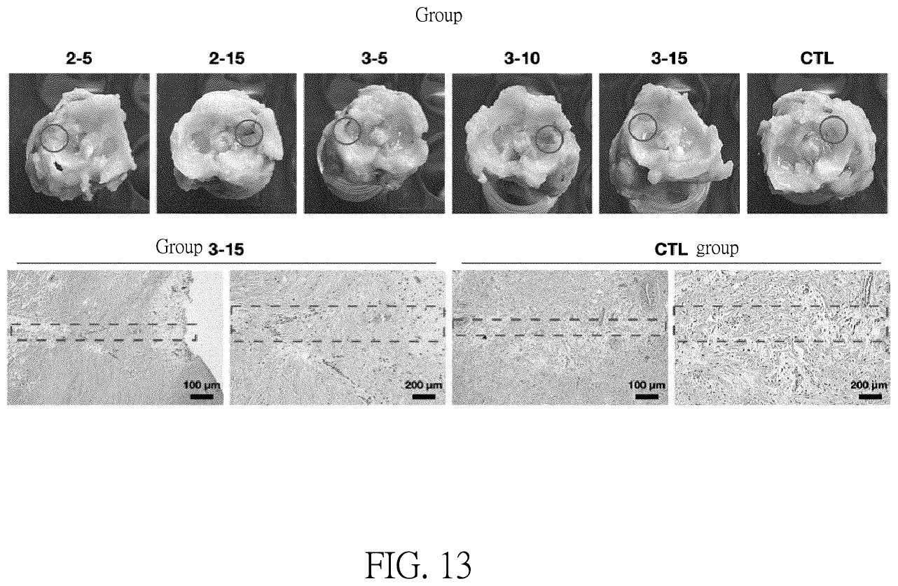

The weight change results are used to represent the degradation behavior of the tissue adhesives of the present disclosure. FIG. 5 shows the weight change results after normalization. At the first measurement point (3 days), group 3-15 demonstrates the highest remaining weight for 98.4%, and group 3-5 presents the lowest remaining weight for 80.0%. After 21 days of immersion, group 2-15 exhibits the highest remaining weight for 64.0%, and group 3-5 exhibits the lowest remaining weight for 34.0%. To design a suitable degradation profile of tissue adhesives, one must consider the healing rate of the target tissue. Generally, the complete healing time of a torn meniscus requires around 8 weeks postoperatively, and the tissue adhesive has to remain functional at the site of the target tissue and provide mechanical properties during that period.

### Tissue Adhesive Properties of Tissue Adhesives

Tissue adhesive properties of the tissue adhesives of the present disclosure are analyzed by placing groups 2-5, 2-15, 3-5, 3-10, and 3-15 between two pieces of porcine meniscus for a lap shear stress test. Left panels of FIG. 6 show the setting for the lap shear stress test, combining meniscus pieces and nylon sutures with hooks. The adhesion area is around 25 mm² with 5 μl of the tissue adhesive of the present disclosure. The results of the lap shear stress test show that the highest stress occurred in group 3-5 with 799.1 kPa of shear stress. At the opposite end, the lowest stress occurred in group 3-10 with 143.9 kPa of shear stress (right panel of FIG. 6). The primary requirements of tissue adhesive properties of the tissue adhesives of the present disclosure for a meniscus are to resist physiological stresses and stabilize the torn meniscus tissue to prevent gap formation. The tissue adhesive which can provide stresses of 50 kPa to 100 kPa to hold a torn meniscus together is able to meet the requirements of the tissue adhesive.

### Cell Viability of SW1353 Chondrocytes

Cell viability of SW1353 chondrocytes is determined with the MTT assay, and results are presented in the panels of FIG. 7. After 1 day of incubation, cell viabilities of all groups show no differences. However, at 3 and 7 days of incubation, group 2-5 presents the lowest cell viability, which is much lower than that of the control group (CTL). The primary materials of tissue adhesives of the present disclosure are chitosan-based hydrogel and dextran solution, and have been proven to be biocompatible.

### Migration Capacity Tests and Chemotactic Effects of Tissue Adhesives on SW1353 Chondrocytes

Cell migration and cell chemotactic effects are important issues to recruit surrounding cells to the tear site for meniscus tear repair. Upper left panel of FIG. 8 shows the migration of SW1353 chondrocytes after making the initial scratch and after 24 hr of incubation. The reduction in the size of the gap due to the migration of SW1353 chondrocytes is measured using ImageJ software. Compared to the control group, groups 2-5 and 3-5 exhibit significantly reduced gaps after 24 hr (upper middle panel of FIG. 8), with the distance having been reduced by 108.1±27.3 μm in group 2-5 and by 107.9±28.7 μm in group 3-5. On the other hand, the chemotactic effect of SW1353 chondrocytes is tested through a microchannel chip platform. Cells in all groups are migrated and the migration in groups 2-5, 2-15, 3-5, 3-10, and 3-15 is much higher than that of in the control group after 24 and 48 hr of incubation for different levels (lower panels of FIG. 8). After 24 hr of incubation, the migration distance of experimental groups (i.e., groups 2-5, 2-15, 3-5, 3-10, and 3-15) are all higher than that of the control group, with the highest migration distance in group 3-10 at 153±63 μm (upper right panel of FIG. 8). After 48 hr of incubation, the highest migration distance is 202±40 μm appears in group 3-10. Interestingly, the trend of migration distances in each group in the microchannel test for the chemotactic effect test is similar to the wound-healing test for the migration capacity. Cells may be able to move and reach a suitable position in the given environment to perform the function thereof and further form new tissues; therefore, cell migration is a critical process. In at least one embodiment of the present disclosure, cell migration speed and capacity induced by tissue adhesive of the present disclosure may play a role in reducing healing times.

### GAG Synthesis of SW1353 Chondrocytes

The level of GAG synthesis of SW 1353 chondrocytes is detected by staining with alcian blue. Left panels of FIG. 9 show images of alcian blue staining after 7 days of incubation. Significant differences in the stained areas may be observed among experimental groups (i.e., groups 2-5, 2-15, 3-5, 3-10, and 3-15) and control group. Quantitative results at 7 days and 14 days are demonstrated in the middle and right panels of FIG. 9, respectively. At the early period of 7 days of incubation, groups 3-5, 3-10, and 3-15 exhibit significantly higher absorbances, demonstrating that these three groups synthesized more GAG compared to that of the control group. After 14 days of incubation, all groups showed similar GAG expressions except group 2-5 which is significantly higher than the other groups.

In some embodiments, the meniscus tissue mainly contains 72% water, 22% collagens, and 0.8% GAG. In the primary weight-bearing area of meniscal horns and the inner half of menisci, the highest GAG concentrations support bulk compression. Therefore, the GAG content may reflect the healing degree.

### Histological Analysis of Adhesive Menisci in the Ex Vivo Test

After 30 days of incubation, adhesive menisci of porcine with/without suturing are first sectioned and stained. Upper panel of FIG. 10 illustrates H&E staining of adhesive meniscus. Stained images show that groups 2-15, 3-10, and 3-15 demonstrate different levels of adhesion on the meniscus. Moreover, group 2-15 presents superior adhesion under with and without suturing conditions. In addition, groups 3-10 and 3-15 also show desirable adhesion under with and without suturing conditions.

On the other hand, collagen fibers can clearly be seen with the red marker stained by Sirius red (Lower panel of FIG. 10). Group 2-15 with/without suturing and group 3-15 with suturing generate collagen fibers at the meniscus and tissue adhesive boundary. It is noteworthy that the adhesive menisci in group 2-15 under without suturing condition and group 3-15 under with suturing condition generate a notable amount of collagen fibers on both sides of the meniscus-tissue adhesive boundary. In some embodiments, the meniscus has three different collagen structures in three distinct layers: a lamellar layer underneath, a middle layer formed with different sizes of collagen fiber bundles, and an upper layer formed by a dense, randomly oriented collagen fiber network. Regardless of layer of the meniscus, collagen fibers provide mechanical strength to the meniscus tissues.

### ToF-SIMS Profile Images of Adhesive Menisci in the Ex Vivo Test

ToF-SIMS detects collagen fragments with the positive model for C₂H₆N⁻, C₄H₈N⁺, and C₄H₈NO⁺ signals to elucidate the collagen distribution on the adhesive meniscus with suturing (FIG. 11). The dotted box points to adhesion of two sides of the meniscus. Groups 2-5, 3-10, and 3-15 exhibit a clear gap, which means that no collagen is generated in the interfaces between the two pieces of the meniscus. However, collagen signals appear between the interface of the two meniscus pieces in groups 2-15 and 3-5 with suturing. Notably, in the interface of the 3-5 group, suturing cannot clearly be seen, which indicates that a large amount of collagen has formed between the interface. The present disclosure provides a biological technique by using the tissue adhesive to develop collagen tissues between two tear/lesion edges of the meniscus may promote healing of the torn meniscus tissue.

### Extrusion Analysis of Repaired Menisci by MRI

Load transmission and shock absorption are two major biomechanical functions of the meniscus; thus, the stability of repaired menisci is a critical issue to determine the repair situation. Upper panels of FIG. 12 show MR images of a repaired meniscus from New Zealand rabbits at weeks (wk) 2 and 4 postoperatively. After measuring the meniscal body extrusion and meniscal coronal extrusion from MR images of the upper panels of FIG. 12 by ImageJ software, the relative percentage of extrusion is calculated according to equation 2. The calculated relative percentages of extrusion at weeks 2 and 4 postoperatively are shown in lower left panel of FIG. 12 and lower middle panel of FIG. 12. All groups of the tissue adhesive of the present disclosure exhibit less meniscus extrusion at weeks 2 and 4 postoperatively; however, group 3-5 displays relatively less extrusion compared to that of the other tissue adhesive groups. In addition, the level of meniscus extrusion between weeks 2 and 4 is not significant in all groups (lower right panel of FIG. 12). The results indicate that meniscus extrusion is not affected by time progression, but also tissue adhesion may effectively decrease the degree of meniscus extrusion, which means that the tissue adhesive may provide good adhesion onto the torn meniscus.

In some embodiments, the lateral meniscus is more mobile than the medial meniscus. The load acting on the meniscus may generate multiple types of stresses, such as compressive, tensile, and shear forces, and some of the stress is converted into hoop stresses. These multiple directions of stress are countered by the integrity of the collagen bundles in the meniscus and meniscus roots which are attached. The meniscus may extrude when the resistive structures are disrupted. The present disclosure selects the highly mobile region of the meniscus as a tear and repair site to prove the adhesion ability of tissue adhesives and the ability to assist the regenerative capacity.

### Repaired Meniscus Profile and Histological Analysis

The repaired meniscus has to be harvested after 4 weeks postoperatively. Before histological sectioning, macro views of repaired menisci in different groups of tissue adhesives are captured with a camera and are shown in upper panels of FIG. 13. The defect and repaired site are indicated by the red circle. Groups 2-15, 3-5, and 3-10, and the control group display severely torn menisci at 4 weeks postoperatively; however, the repaired meniscus in group 3-15 presents a regenerated situation, and the profile of the meniscus is observed to be complete in the macro view. Afterwards, meniscus tissues are sectioned and stained by H&E staining. Lower panels of FIG. 13 show the repaired and regenerated meniscus tissues in the group 3-15 and control group. Group 3-15 shows dense newly formed tissue at the defect site. Cells have been induced to the repair site and showed a specific regular arrangement. On the other hand, the control group shows a relatively loose organizational structure at the defect site, and cells are distributed in the surrounding tissue without a particular arrangement. Cell migration is an important issue in the repair and development of injury and disease of various tissues. After a meniscal tear, mature meniscal cells from a healthy and undamaged region will migrate through dense, aligned collagen fibers to initiate repair at the wound site.

In at least one embodiment of the present disclosure, tissue adhesives are widely used to adhere and seal trauma and wounds of skin or organs. The present disclosure develops a tissue adhesive with chitosan-based hydrogel and dextran solution, and provides insights into the development of a high-performance tissue adhesive material to offer good biocompatibility and also high adhesion strength, which can promote cell migration and chemotaxis ability, and improve the chondrogenesis ability. The tissue adhesive may have a great potential for meniscus repair and regeneration applications and can be extended to diverse applications beyond meniscus repair.

Those skilled in the art will readily observe that numerous modifications and alterations of the composition and method may be made while retaining the teachings of the invention. Accordingly, the above disclosure should be construed as limited only by the metes and bounds of the appended claims.

What is claimed is:

1. A biocompatible composition, comprising a chitosan-based hydrogel and a dextran solution.

2. The biocompatible composition of claim 1, wherein the chitosan-based hydrogel is crosslinked with the dextran solution.

3. The biocompatible composition of claim 1, wherein the chitosan-based hydrogel is 2% to 90% by weight of the biocompatible composition.

4. The biocompatible composition of claim 1, wherein the chitosan-based hydrogel comprises:
a chitosan; and
a hydrochloride salt.

5. The biocompatible composition of claim 4, wherein
the chitosan is 2% to 85% by weight of the biocompatible composition; and
the hydrochloride salt is 0.1% to 5% by weight of the biocompatible composition.

6. The biocompatible composition of claim 1, wherein the dextran solution is 2% to 20% by weight of the biocompatible composition.

7. The biocompatible composition of claim 1, wherein the dextran solution comprises:
a dextran;
a sodium metaperiodate solution;
a sodium bicarbonate solution; and
a potassium iodide solution.

8. The biocompatible composition of claim 7, wherein
the dextran is 5% to 10% by weight of the biocompatible composition;
the sodium metaperiodate solution is 0.1% to 10% by weight of the biocompatible composition;
the sodium bicarbonate solution is 0.1% to 5% by weight of the biocompatible composition; and
the potassium iodide solution is 0.05% to 20% by weight of the biocompatible composition.

9. The biocompatible composition of claim 7, wherein the ratio of dextran solution to sodium metaperiodate solution is 1:0.1 to 1:8.

10. A method for preparing the biocompatible composition of claim 1, comprising:
reacting the chitosan-based hydrogel with the dextran solution via a cross-linking reaction.

11. The method of claim 10, wherein the cross-linking reaction has a gelation time of 0.5 min to 10 min.

12. The method of claim 10, wherein the chitosan-based hydrogel is 2% to 90% by weight of the biocompatible composition.

13. The method of claim 10, further comprising preparing the chitosan-based hydrogel by mixing a chitosan powder and an ethanolic HCl.

14. The method of claim 10, wherein the dextran solution is 2% to 20% by weight of the biocompatible composition.

15. The method of claim 10, further comprising preparing the dextran solution by mixing a dextran, a sodium metaperiodate solution, a sodium bicarbonate solution, and a potassium iodide solution.

16. The method of claim 15, wherein the ratio of dextran solution to sodium metaperiodate solution is 1:0.1 to 1:8.

17. A method of tissue adhesion, comprising administering the biocompatible composition of claim 1 to a subject in need thereof.

18. The method of claim 17, wherein the biocompatible composition is topically administered to the subject.

19. The method of claim 18, wherein the biocompatible composition is topically administered to the cartilage tissue of the subject.

20. The method of claim 19, wherein the cartilage tissue is a fibrocartilaginous structure.
